(19) United States
(12) Patent Application Publication
(10) Pub. No.: US 2016/0022209 A1
(43) Pub. Date: Jan. 28, 2016

(54) SYSTEMS AND METHODS FOR TRACKING HYDRATION

(71) Applicant: HYDR8 Inc., Saratoga, CA (US)

(72) Inventor: Daniel J. Fraisl, Saratoga, CA (US)

(21) Appl. No.: 14/754,599

(22) Filed: Jun. 29, 2015

Related U.S. Application Data
(60) Provisional application No. 62/018,079, filed on Jun. 27, 2014.
(60) Provisional application No. 62/162,510, filed on May 15, 2015.

(51) Int. Cl. A61B 5/00; A61B 5/11; A63B 24/00

(52) U.S. Cl. CPC A61B 5/4875; A63B 24/0062; A61B 5/4205; A61B 5/6887; A61B 5/7278; A61B 5/0024; A61B 5/7282; A61B 5/486; A61B 5/1118; A63B 2024/0065; A61B 2560/0462; A61B 2562/0247; A61B 2560/0475; A61B 2562/0271; A61B 2562/029; A61B 2562/0219; A61B 2560/0209

(57) ABSTRACT

Devices, systems, and methods are provided for monitoring hydration of a user that include a fluid container including one or more sensors, e.g., pressure sensors, accelerometers, and the like, for identifying when the user consumes fluid from the container and/or determining the volume of fluid consumed. The fluid container may communicate with a mobile electronic device to monitor at least one of volume and frequency of fluid consumption and provide an output to the user related to compliance with hydration recommendations. Optionally, the system may also include a fitness tracker for monitoring one or more aspects of the user's physical activity; and the mobile device may monitor hydration of the user based at least in part on the user's physical activity.

### RELATED APPLICATION DATA

The present application claims benefit of co-pending provisional applications Ser. No. 62/018,079, filed Jun. 27, 2014, and 62/162,510, filed May 15, 2015, the entire disclosures of which are expressly incorporated by reference herein.

### FIELD OF THE INVENTION

The present invention relates generally to devices for providing fluids to individuals, and more particularly to systems and methods for tracking and/or monitoring hydration of an individual consuming fluid within a fluid container.

### BACKGROUND

As portable electronic devices have proliferated and become increasingly powerful and capable, the features for which they are commonly used have shifted. As pocket-sized devices have transitioned from being purely communication devices, to becoming content-consumption devices, to becoming content-creation devices, and now to becoming health-tracking devices, users have also transitioned towards becoming prodigious health data consumers obsessed with tracking and optimizing their personal health data. The advent of advanced sensors and wearable devices (such as the FitBit, Nike Fuel Band, Jawbone UP, and a host of other health tracking devices) is accelerating the amount and type of data that users wish to capture, track, and share.

Unfortunately, for many people one important health metric that cannot be easily measured and tracked using portable electronic devices today is hydration. According to the CDC, as of 2013, among adults aged twenty to fifty (20-50), 43% of men and 41% of women failed to meet the daily water intake recommended by the US Institute of Medicine. People aged fifty to seventy (50-70) were less likely to meet the guidelines, and the numbers were even worse among older adults—roughly 95% of men and 83% of women seventy one and older don't drink enough water. Further, for athletes, the military and first-responders maintaining proper hydration levels can be a life or death matter.

Accordingly, systems and methods for monitoring and/or improving hydration of individuals would be useful.

### SUMMARY

The present invention is directed to devices for providing fluids to individuals, and more particularly to fluid containers that facilitate tracking and/or monitoring hydration of an individual consuming fluid within the fluid container. Further, the present invention is directed to systems and methods for tracking and/or monitoring hydration of an individual consuming fluid within a fluid container, e.g., using a mobile electronic device communicating with one or more sensors on the fluid container and/or communicating with a fitness tracker used by the individual.

Systems are provided herein, which may be implemented in one or more fluid dispensing containers. In an exemplary embodiment, the system may allow a user to measure and track their hydration level by automatically measuring and tracking the volume of fluid orally consumed from the fluid dispensing container. The system may employ one or more sensors to determine the volume of fluid that flows from the fluid dispensing container. In an exemplary embodiment, the fluid container may include air flow volume measurement or air pressure measurement to determine the proportional liquid flow volume that is displaced by the air flowing into the container. In alternative embodiments, the fluid container may include one or more other liquid measurement sensors, such as a pressure sensor in the bottom of the container to correlate height of fluid within the container to volume of fluid, an accelerometer to correlate movement of fluid within the container to mass and volume of the fluid, or an ultrasonic sensor to correlate height of fluid within the container to volume of fluid. Such sensors may be included in a closed-loop hydration tracking system, e.g., including a mobile electronic device and/or a fitness tracker.

Also provided herein are methods for determining calculating the volume of liquid consumed by a user, reducing the physical size of the system, conserving the amount of power necessary to operate the system, and/or transmitting data between the system and a mobile device, server, and/or external computer. In both the systems and methods, volume measurement may be combined with data from a fitness tracker and/or other device providing data regarding physical activity of the user, environmental conditions at the user's location, physical characteristics of the user, and/or external data sources, to enhance operation.

In accordance with one embodiment, a device is provided for monitoring hydration that includes a fluid container comprising a container body defining an interior region and a removable top for enclosing the interior region; a one-way valve in the top biased to a closed position for sealing an opening in the top communicating with an exterior of the fluid container and movable to an open position when the interior region experiences a negative pressure to allow air to flow into the interior region from the exterior via the opening; a drinking tube extending from the top into interior region; an air pressure sensor within the fluid container at a location such that the sensor is exposed to air when the fluid container is filled with a hydration fluid; and a processor coupled to the sensor and configured to detect a first predetermined pressure change within the interior region corresponding to a drink start event when a user begins to drink hydration fluid from the interior region via the drinking tube, the processor further configured to determine a volume of hydration fluid consumed by the user based on changes in air pressure within the interior region.

In accordance with another embodiment, a device is provided for monitoring hydration that includes a fluid container comprising a container body including a side wall and a bottom wall defining an interior region and a removable top for enclosing the interior region; a drinking port in the top for consuming hydration fluid contained within the fluid container; a liquid pressure sensor mounted to the bottom wall within the interior region; an inclinometer mounted to the fluid container; and a processor coupled to the pressure sensor to acquire pressure data to determine a height of hydration fluid within the interior region and coupled to the inclinometer to acquire orientation data to determine an angular orientation of the fluid container when the height is determined, the processor further configured to determine a volume of hydration fluid consumed by the user based on changes in the height of the hydration fluid air pressure within the interior region corrected by the angular orientation.

In accordance with still another embodiment, a device is provided for monitoring hydration that includes a fluid container comprising a container body including a side wall and a bottom wall defining an interior region and a removable top for enclosing the interior region; a drinking port in the top for consuming hydration fluid contained within the fluid container; an accelerometer mounted to fluid container; and a processor coupled to the accelerometer to acquire motion data related to motion of the fluid container, the processor configured to incorporate the motion data into a mass analysis algorithm to determine a change in mass of the fluid container and hydration fluid therein and determine a volume of hydration fluid consumed by the user.

In accordance with yet another embodiment, a device is provided for monitoring hydration that includes a fluid container comprising a container body including a side wall and a bottom wall defining an interior region and a removable top for enclosing the interior region; an ultrasonic sensor mounted within the interior region and oriented towards an upper surface of fluid when the fluid is placed in the container; an inclinometer mounted to the fluid container; and a processor coupled to the ultrasonic sensor to acquire ultrasonic data related to ultrasonic signals transmitted by the ultrasonic sensor and reflected off of the upper surface of the fluid, the processor coupled to the inclinometer to acquire orientation data to determine an angular orientation of the fluid container when ultrasonic data is acquired, the processor further configured to determine a height of hydration fluid within the interior region based at least in part on the ultrasonic data corrected by the angular orientation and determine a volume of hydration fluid consumed by the user based on changes in the height of the hydration fluid within the interior region.

In accordance with another embodiment, a device is provided for monitoring hydration that includes a fluid container comprising a container body including a side wall and a bottom wall defining an interior region and a removable top for enclosing the interior region; an air flow switch in air flow passageway communicating between the interior region and an exterior region outside the container; a processor coupled to the air flow switch for acquiring air flow data to detect flow of air through the air flow passageway to identify the beginning of a drink event, the processor configured to operate electronic components of the device in a low-power state before a drink event to conserve energy and configured to activate the electronic components in a full-power state when a drink event is identified; and one or more sensors coupled to the processor for determining a volume of fluid consumed during the drink event.

In accordance with still another embodiment, a device is provided for monitoring hydration that includes a fluid container comprising a container body including a side wall and a bottom wall defining an interior region and a removable top for enclosing the interior region; a motion sensor carried by the container; a processor coupled to the motion sensor for acquiring motion data to identify the beginning of a drink event, the processor configured to operate electronic components of the device in a low-power state before a drink event to conserve energy and configured to activate the electronic components in a full-power state when a drink event is identified; and one or more sensors coupled to the processor for determining a volume of fluid consumed during the drink event.

In accordance with yet another embodiment, a system is provided for managing hydration of a user that includes a fluid container comprising one or more sensors for identifying when the user consumes fluid from the container and a container communication interface; a fitness tracker for monitoring one or more aspects of the user's physical activity; and a mobile device comprising a mobile device communication interface for communicating with the one or more sensors of the fluid container via the container communication interface to monitor at least one of volume and frequency of consumption of hydration fluid from the container and for communicating with the fitness tracker to monitor one or more aspects of the user's physical activity, and a mobile device processor configured to monitor hydration of the user based at least in part on the hydration fluid consumed from the container and the user's physical activity and provide an output on an output device to the user related to compliance with hydration recommendations.

In accordance with another embodiment, a method is provided for monitoring hydration of a user that includes providing a fluid container comprising a container body, a top, a pressure sensor mounted to a bottom wall of the container body within an interior of the fluid container, and a processor communicating with the pressure sensor; adding hydration fluid to the container body; securing the top to the container body to enclose the interior; and consuming at least a portion of the hydration fluid within the fluid container, thereby triggering the processor to acquire pressure data from the pressure sensor and determine a height of hydration fluid within the fluid container and compare the height with an earlier height of hydration fluid to determine a consumed volume of the at least a portion of the hydration fluid. In one embodiment, an inclinometer may be provided on the fluid container for providing orientation data related to the angular orientation of the fluid container, and the processor may correct the height of hydration fluid based at least in part on orientation data acquired from the inclinometer.

In accordance with still another embodiment, a method is provided for monitoring hydration of a user of a fluid container including one or more sensors that includes receiving data from the one or more sensors indicating that the container is in motion or a drink event has occurred; determining an amount of fluid consumed from the fluid container during the drink event; acquiring activity data from a fitness tracker worn by the user; and comparing the amount of fluid consumed with a hydration threshold to determine whether the user has consumed sufficient fluid based at least in part on the activity data acquired from the fitness tracker.

In accordance with another embodiment, a method is provided for monitoring hydration of a user of a fluid container including a motion sensor that includes receiving a signal from the motion sensor indicating that a drink event has begun; based on the signal, transitioning electronic components of the consumption tracking system from a low-power state to a full-power state; and acquiring sensor data from one or more sensors on the fluid container and determining, at the consumption tracking system, an amount of fluid consumed from the fluid container during the drink event based at least in part on the sensor data.

In accordance with yet another embodiment, a method is provided for monitoring hydration of a user of a fluid container including an air flow switch communicating between an interior region of the container and an exterior region outside the container and coupled to a consumption tracking system, the method including receiving a signal from the air flow switch when air begins flowing through the air flow passageway indicating that a drink event has begun; based on the signal, transitioning electronic components of the consumption tracking system from a low-power state to a full-power state; and acquiring sensor data from one or more sensors on the fluid container and determining, at the consumption tracking system, an amount of fluid consumed from the fluid container during the drink event based at least in part on the sensor data.

In accordance with still another embodiment, a method is provided for monitoring hydration of a user of a fluid container including one or more sensors and fluid within an interior region of the container that includes receiving one or more signals from the one or more sensors indicating that a drink event has occurred; acquiring height data from the one or more sensors on the fluid container related to a height of fluid within the container; acquiring orientation data from the one or more sensors on the fluid container to determine an angular orientation of the fluid container when the height data is acquired; and determining a height of fluid within the interior region based at least in part on the height data corrected by the angular orientation and determining a volume of fluid consumed during the drink event based on changes in the height of the hydration fluid within the interior region.

In accordance with another embodiment, a hydration management system is provided that includes a plurality of individual hydration management systems, each individual system comprising a fluid container including one or more sensors configured to output real-time hydration data indicative of volume of fluid in the container consumed by a user; a fitness tracker configured to monitor one or more characteristics of the user's physical activity; and a mobile electronic device configured to communicate with the fluid container to acquire the hydration data from the one or more sensors to monitor fluid consumption by the user, configured to acquire activity data from the fitness tracker, and configured to provide output to the user regarding the user's hydration. The system also includes a hydration management server configured to communicate with each mobile electronic device of the individual hydration management systems and operative to receive, analyze, and/or aggregate the hydration data, activity data, and user hydration from each of the individual hydration management systems.

In accordance with still another embodiment, a method is provided for hydration management using a plurality of individual hydration management systems, each individual system comprising a fluid container including one or more sensors configured to output real-time hydration data indicative of volume of fluid in the container consumed by a user; a fitness tracker configured to monitor one or more characteristics of the user's physical activity; and a mobile electronic device configured to communicate with the fluid container to acquire the hydration data from the one or more sensors to monitor fluid consumption by the user, configured to acquire activity data from the fitness tracker, and configured to provide output to the user regarding the user's hydration. The method includes receiving at a hydration management server hydration data, activity data, and user hydration from each mobile electronic device of the individual hydration management systems; generating a hydration database combining the hydration data and activity data with physical characteristics data of the respective individuals; generating recommended hydration thresholds based at least in part on the hydration database; and communicating recommended hydration thresholds to the individual hydration management systems such that each mobile electronic device may provide outputs to the user regarding the user's hydration based at least in part on the recommended hydration thresholds.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1A is a cross-sectional side view of an exemplary hydration tracking system including an air pressure sensor in a fluid container for measuring fluid consumed.

FIG. 1B is a schematic showing exemplary components that may be included in the fluid container of FIG. 1B to monitor hydration of a user of the fluid container.

FIGS. 2A-2C are cross-sectional side views of alternative embodiments of hydration tracking systems including a pressure sensor in a fluid container for measuring fluid consumed.

FIGS. 3A-3C are cross-sectional side views of alternative embodiments of hydration tracking systems including an accelerometer coupled to a fluid container for measuring fluid consumed.

FIG. 4 is a schematic showing an exemplary feedback system that may be used to monitor hydration of one or more individuals using a hydration system, such as those shown in FIGS. 1-3.

FIG. 5 is a flowchart showing an exemplary method for monitoring hydration of an individual.

FIG. 6 is a schematic showing an exemplary network architecture including a plurality of hydration tracking systems, such as that shown in FIG. 4.

FIG. 7 is a schematic showing an exemplary embodiment of a mobile electronic device that may be included in the hydration tracking system of FIG. 4.

FIG. 8 is a cross-sectional view of another embodiment of a consumption tracking device that may be provided on a fluid container.

FIG. 9 is a schematic showing an exemplary processing subsystem that may be included in the consumption tracking device of FIG. 8.

FIG. 10 is a flowchart showing an exemplary method for using the consumption tracking device of FIG. 8.

### DETAILED DESCRIPTION OF THE EXEMPLARY EMBODIMENTS

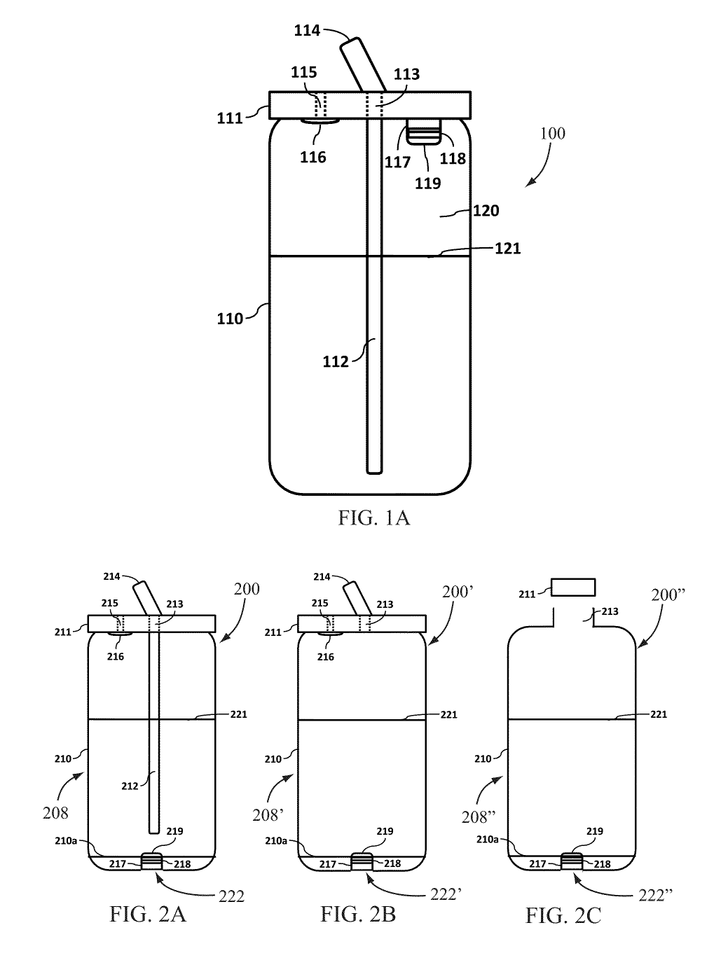

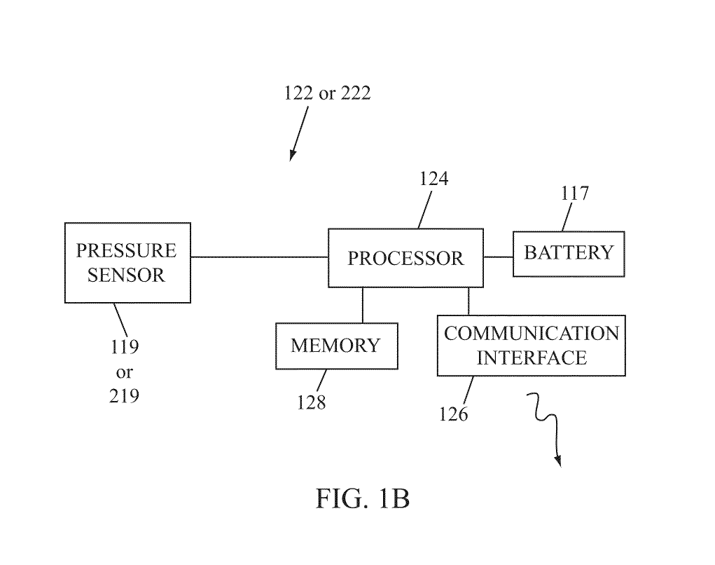

Turning to the drawings, FIGS. 1A and 1B illustrate an example of a hydration tracking system or device 100 that generally includes a fluid container 108 and a fluid consumption tracking device 122 for monitoring fluid consumption of a user of the system 100. The container 108 may include a container body or housing 110, and one or more of a removable top or cap 111, straw 112, connecting tube 113, drinking tube (bite or open) 114, primary air flow passageway 115, and/or a one-way air valve 116. The container 108 may hold a liquid or other fluid 121 for consumption, which in turn results in an air chamber 120 above the liquid 121 when the top 111 is placed on the container body 110. A user of the container 108 consumes liquid from the fluid container 108, e.g., water, electrolyte beverage, energy drink, and the like, through the drinking tube 214.

The consumption tracking device 122 may include an air pressure sensor 119 placed within the air chamber 120 of the container 108, e.g., above the maximum level of the liquid 121, a replaceable or rechargeable battery or other power source 117 for providing electrical power to components of the consumption tracking device 122, and one or more electronic components 118. For example, as shown in FIG. 1B, the consumption tracking device 122 may include a processor unit 124, memory resource(s) 128 for storing data and instructions executable by the processor 124, and/or a communication interface 126, e.g., a wireless transmitter or transceiver, for communicating with a remote device, e.g., a mobile electronic device, external computer, and the like (not shown). Optionally, the consumption tracking device 122 may include one or more additional sensors including an accelerometer, inclinometer, gyroscope, temperature sensor, and/or humidity sensor (not shown), if desired, as described elsewhere herein. In another option, the liquid consumption tracking device 122 may also be electronically connected to an output device, e.g., one or more LEDs and/or a visual display, a speaker, and the like (not shown), mounted anywhere on or in the container body 110 or container top 111, e.g., to provide various types of feedback to the user of the system 100, as described elsewhere herein.

The electronic components of the consumption tracking device 122 may be enclosed in a housing (not shown) that may be designed to easily connect to the container body 110, top 111, or straw 112. The housing may be designed to be water proof and dishwasher safe to protect the electronic components from exposure to the liquid in the container 108 and/or to water or steam during cleaning of the hydration tracking system 100.

The consumption tracking device 122 may provide consumption data based at least in part on the air pressure sensor 119. For example, the processor 124 may acquire pressure data from the air pressure sensor 119 to measure the flow of air into the container 108 through the primary air flow passageway 115, e.g., by measuring, over time, the air pressure changes in the air chamber 120 above the liquid 121 residing in the container 108.

When a user of the system 100 draws liquid up the straw 112, an air pressure drop occurs in the air chamber 120 above the liquid 121 in the container 108. This air pressure drop in turn causes the one-way air valve 116 to open, which allows a volume of air to flow into the chamber 120 that is proportional to the liquid flowing out of the container 108. By measuring the volume of air that flows into the container 108 during a drink, the consumption tracking device 122 may substantially simultaneously measure the volume of liquid flowing out of the container 108.

The air pressure sensor 119 may be locally exposed to the primary air flow passageway 115 while not directly connected to either the primary air flow passageway 115 or the one-way air valve 116. In particular, the air pressure sensor 119 may be spaced sufficiently from the air flow passageway 115 such that the liquid consumption device 122 does not affect either the liquid flow out of the container 108 or the air flow into the container 108. In addition, this separation enables the addition of the liquid consumption device 122 to conventional containers without modifying the container.

When a user of the consumption tracking device 122 is not drinking, the processor 124 may acquire pressure data from the air pressure sensor 119 periodically or otherwise intermittently to measure the internal ambient air pressure within the container 108. This measured internal ambient air pressure may, in turn, be used to calculate the external ambient air pressure using a conversion formula based on the mechanical structure and size of the primary air flow passageway 115 and/or one-way air valve 116, as is known to those of skill in the art. In this manner, the external ambient air pressure may be calculated as an input into the calculation of air volume flowing through the primary air flow passageway 115 and into the container 108 while a user is drinking.

Also, since external ambient air pressures outside the container 108 may arbitrarily and constantly change, the processor 124 may acquire pressure data from the air pressure sensor 119 to recalculate and/or store a recent external ambient air pressure value before a drink event starts and/or after a drink event is complete.

During a drink event, the processor 124 may acquire pressure data from the air pressure sensor 119 to calculate the volume of air that flows into the container 108 through the primary air flow passageway 115 using Bernoulli's equation:

Q=Cf*A*sqrt((2*(p(t)1−p2(t)))/density)

where Q is the air flow rate, A is the surface area of the vent hole, p1(t) is the external ambient air pressure at time t and p2(t) is the chamber air pressure at time t. The air flow volume during a time interval may then be calculated from the air flow rate during that time interval by multiplying Q by the time interval. Finally, the total air flow volume during a drink event may then be calculated by summing the individual air flow volumes from each time interval.

To improve the accuracy of the air flow calculation, optionally, a temperature sensor (not shown) may be provided on the container 108 to measure the external temperature at time t and/or a humidity sensor may be provided on the container 108 to measure the external humidity at time t. The processor 124 may then use the temperature and/or humidity measurements to accurately calculate air density at the time of a drink.

Further, to calculate air flow volume, the processor 124 needs to identify when a drink event starts and ends. For example, the processor 124 may acquire pressure data from the air pressure sensor 119 periodically or otherwise intermittently to identify a unique pressure change signature pattern that corresponds to a drink start event and a different unique pressure change signature pattern that corresponds to a drink end event.

The one-way air valve 116 may be biased to a closed position for sealing the passageway 115 in the top 111 yet movable to an open position when the interior air chamber 120 experiences a negative pressure relative to the exterior of the container 108 to flow into the air chamber 120. Thus, during a drink event, the one-way air valve 116 opens and closes as the difference between external air pressure and internal air pressure changes. Because of this phenomena, the term A in the flow rate calculation equation constantly changes as a function of the difference between internal air pressure and external ambient air pressure. The processor 124 may include a function that predicts the value of A based upon the instantaneous pressure difference between external and internal ambient.

Optionally, the system 100 may include a motion sensor (not shown) coupled to the consumption tracking device 122, e.g., mounted on the container body 110 or container top 111 (on the exterior or interior, as desired). The processor 124 may be coupled to the motion sensor to operate one or more electronic components of the system 100, e.g., the processor 124 itself, the air pressure sensor 119, and/or communication interface 126, at a low-power state for the purpose of reducing overall energy consumption of the system 100 when the system 100 is inactive. For example, when the container 108 is moved, e.g., immediately before a drink event, the processor 124 may identify a motion signal from the motion sensor indicating that the container 108 has moved, and the processor 124 may activate the components of the consumption tracking device 122 and enable the system 100 to transition to a full-power state. The consumption tracking device 122 may remain in the full-power state only while the container 108 is in motion, i.e., while motion signals from the motion sensor indicate the container 108 is in motion, or alternatively the consumption tracking device 122 may remain in the full-power state for a predetermined time, e.g., corresponding to the maximum anticipated duration of a drink event, and then acquire additional motion data to reconfirm whether the container 108 is still in motion.

Thus, during use, the system 100 may remain in a low-power state until the processor 124 receives a motion signal from the motion sensor, whereupon the system 100 may be converted to the full-power state. Once in the full-power state, the processor 124 may acquire pressure data from the air pressure sensor 119 until the processor 124 identifies that a drink event has begun, e.g., based on a first predetermined pressure change pattern or other indicator. Once the processor 124 identifies that a drink event has begun, the processor 124 may acquire pressure data from the air pressure sensor 119 during the drink event, discontinuing upon completion of the drink event, e.g., based on a second predetermined pressure change pattern or other indicator. The processor 124 may then determine the volume of fluid consumed based at least in part on the pressure data (and optionally other data, such as temperature and/or humidity data to compensate for the air density), as described above.

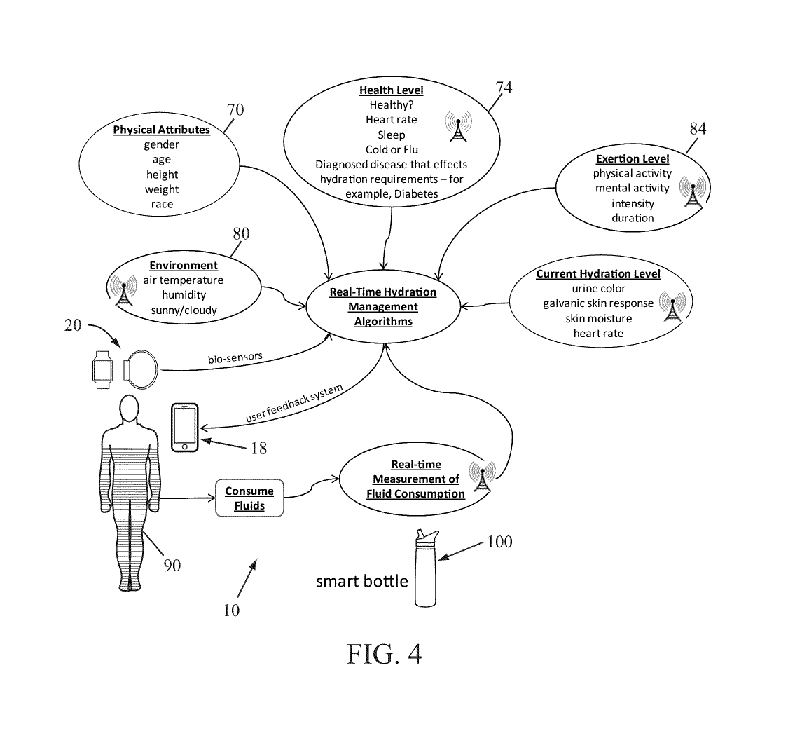

The resulting volume consumption data may be stored in memory 128 within the consumption tracking device 122 and/or communicated to another remote electronic device, e.g., a mobile device (not shown, see, e.g., device 18 in FIG. 4) operating a software or other hydration and/or fitness application, via the interface 126, as described elsewhere herein. Optionally, the processor 124 may use consumption data to provide an output to the user, e.g., to provide an indication whether the user has consumed sufficient hydration fluid, e.g., based on one or more of the user's physical characteristics, physical activity, environmental conditions, and the like, as described elsewhere herein. For example, the processor 124 may be coupled to an output device on the container 108, such as one or more LEDs or a display (not shown) for providing a visual indication or a speaker or other device for providing an audible indication, e.g., when the user has consumed sufficient fluid or needs to consume additional fluid.

In an alternative embodiment, the volume consumption data may be stored in memory 128 until the processor 124 receives a request from a remote device (e.g., the electronic device 18 shown in FIG. 4), whereupon the consumption data may be transmitted via the communication interface 126 to the remote device. In another alternative, the pressure data (and any other raw data) may be transmitted to the remote device, which may perform any calculations to determine volume consumption and/or any other information. In this alternative, the remote device may provide an output related to the fluid consumption, as described elsewhere herein.

In an alternative embodiment, other sensors may be mounted to the top 111, e.g., within the air chamber 120, for determining the volume of fluid within the container 108. For example, instead of the air pressure sensor 119, the sensor 119 may be an ultrasonic sensor that may be oriented downwardly towards the bottom of the container body 110, e.g., substantially perpendicular to the inner surface of the top 111, i.e., substantially vertically relative to the container body 110. The sensor 119 may be configured to transmit ultrasonic signals from the inner surface towards the upper surface of the fluid 121 within the container 208 and detect signals reflected from the upper surface of the fluid 121. Alternatively, separate ultrasonic transmitter and receivers may be provided (not shown).

The processor 124 may acquire data from the ultrasonic sensor 119, e.g., time delay data based on the difference between the time of transmission of an incident ultrasonic wave or signal and a wave or signal reflected from the upper surface of the fluid 121 back to the sensor 119. Given the known speed of sound within air, the processor 124 may use the difference between the time of the reflected and incident signals to determine a distance between the top 111 and the fluid 121 and correlate the distance to the volume of fluid remaining within the container 208, e.g., after a drink event.

In an alternative embodiment, an ultrasonic sensor may be mounted to the bottom wall of the container body 110, e.g., oriented upwardly towards the top 111, e.g., substantially perpendicular to the bottom wall. In this alternative, the sensor may be configured to transmit ultrasonic signals from the bottom wall upwardly towards the upper surface of the fluid 121 within the container 208 and detect signals reflected from the upper surface of the fluid 121. Given the known speed of sound within the fluid 121, the processor 124 may use the difference between the time of the reflected and incident signals to determine a distance between the bottom wall and the fluid 121 and correlate the distance to the volume of fluid remaining within the container 208.

Optionally, the container 108 may include an inclinometer coupled to the processor 124, which may provide orientation data identifying the orientation of the container 108 relative to vertical, e.g., to compensate for the upper surface of the fluid 121 being non-perpendicular to the vertical axis of the container 108 if the container is oriented non-vertically. Based at least in part on the orientation and the known shape of the container, the processor 124 may determine the volume of fluid remaining within the container 108 after a drink event and determine the volume of fluid consumed during the drink event.

Turning to FIGS. 2A-2C, additional examples of a hydration tracking system 200, 200,′ 200″ are shown that are generally similar to the system 100 although using a different pressure sensor 219.

For example, FIG. 2A illustrates a liquid dispensing container 208 including a container body or housing 210 and one or more of a removable top 211, straw 212, connecting tube 213, drinking tube 214 (e.g., bite or open type), primary air flow passageway 215, and/or a one-way air valve 216. The container 108 may be filled with a liquid or other fluid 221 for consumption, which in turn results in an air chamber 220 above the liquid 221 when the top 211 is placed on the container body 210. A user of the container 210 consumes liquid from the container 210 by drawing liquid through the drinking tube 214.

FIG. 2B illustrates an alternate system 200′ that includes a liquid dispensing container 208′ without a straw member. A user of the container 208′ consumes liquid from the container 208′ by tilting the container 208′ such that liquid is in contact with the drinking tube 214 and then drawing liquid through the drinking tube 214 and/or squeezing the container 208′ to force liquid through the drinking tube 214.

FIG. 2C illustrates an alternate system 200″ that includes a liquid dispensing container 208″ including a drinking orifice 213 without a straw member or drinking tube. A user of the container 208″ consumes liquid from the container 208″ by tilting the container 208″ such that liquid flows out of the drinking orifice 213.

Each of the hydration tracking systems 200-200″ also include a liquid consumption tracking device 222, e.g., including a pressure sensor 219 placed at the bottom of the container 208-208″, e.g., to the inside of a bottom wall 210a of the container 210 such that the pressure sensor 219 is exposed to the liquid 221. In addition, the consumption tracking device 222 may include a replaceable or rechargeable battery 217 and electronic components 218, e.g., which may be similar to the components shown in FIG. 1B, i.e., including a processor unit 124, memory 128 for storing data and instructions executable by the processor unit, and/or a wireless communication interface 126 for communicating with a remote device. Optionally, the systems 200-200″ may also include one or more additional sensors, such as an accelerometer, inclinometer, gyroscope, temperature sensor, and/or humidity sensor. The consumption tracking device 222 may also be electronically connected to an output device, e.g., one or more LEDs or a visual display (not shown) mounted anywhere on the container body 210 or container top 211 to provide various types of feedback to the user of the systems 200-200.″

The electronic components of the consumption tracking device 222 may be enclosed in a housing (not shown) that is designed to easily connect to the bottom of the container body 210. The housing may be designed to be water proof and dishwasher safe to protect the electronic components from exposure to the liquid in the container and/or to water or steam during cleaning of the hydration tracking system.

The consumption tracking device 222 may provide a liquid volume measurement device based at least in part on a pressure sensor 219. The processor 124 may acquire pressure data from the pressure sensor 219 positioned at the bottom 210a of the container body 210 to determine the volume of liquid in the container 208-208,″ e.g., by measuring the pressure of the column of liquid in the container body 210. The pressure of the column of liquid may be calculated by subtracting the ambient air pressure from the total pressure at the bottom of the container body 210.

For example, the pressure within the empty container 208 may be acquired and then any desired compensations may be applied, e.g., based on changes in elevation. Alternatively, the processor 124 may acquire internal air pressure data within the container 208 (e.g., using an air pressure sensor similar to that described above mounted on the top 211), and the pressure data may be adjusted based on the known behavior of the air valve 216 and the container 208. Generally, the air valve 216 may close before the internal pressure equalizes with the external pressure; however, the pressure differential at which the air valve 216 closes is substantially fixed by the design of the air valve 216 and so the pressure differential may be added to the internal pressure to estimate the external ambient pressure immediately after a drink event.

The volume of liquid within the container 208-208″ may be periodically (at regular time intervals) or otherwise intermittently (e.g., at intervals based on identification of a drink event) calculated by measuring the pressure of the column of liquid in the container 208-208″ at each time interval, then calculating any change in liquid volume based on a proportional change in liquid pressure, e.g., similar to the methods disclosed in U.S. Publication No. 2015/0024349, the entire disclosure of which is expressly incorporated by reference herein. However, the systems and methods herein may provide one or more advantages over the devices disclosed in this reference.

For example, to measure liquid volume consumption using the systems 200-200,″ the processor 124 may first measure a starting volume of liquid in the container 208-208,″ e.g., when initially filled with hydration fluid. The processor 124 may recognize unique pressure, acceleration, and/or orientation change signature patterns to automatically sense when liquid is entering the container. For example, the processor 124 may acquire pressure data from the pressure sensor 219 and identify a rapid increase in pressure to correspond to a container filling event. Once the processor 124 identifies that the pressure data substantially stabilizes, the processor 124 may conclude that the container 208-208″ has been filled. In addition or alternatively, a motion sensor (not shown) may be provided on the container 208-208″ and the processor 124 may acquire motion data from the motion sensor and analyze the data to identify a filling event. Optionally, the processor 124 may identify pressure data from the pressure sensor 219 immediately before the filling event as corresponding to ambient air pressure and save this value in memory 128 for subsequent use, if desired.

After liquid filling is complete, the processor 124 may calculate the height H of liquid in the container 208-208″ using the following formula:

h=p/(g*D)

where h is the height of the liquid, p is the measured pressure of the liquid column, g is the gravity constant, and D is the density of the liquid. The processor 124 may then store the height data of the liquid in the container within memory 128 for later use as fluid is consumed from the container 208-208.″ Alternatively, the processor 124 may calculate the initial volume of the fluid from the height data and store the initial volume instead or as well.

For example, once the height of the liquid in the container 208-208″ is known, the volume of the liquid may be computed using a volume function that is dependent on the shape of the container 208-208.″ For example, the volume V function for a cylindrical bottle is:

V=hπr2

where h is the previously computed height of the liquid in the container, pi is a constant, and r is the radius of the bottle.

Once the initial volume of liquid in the container 200-208″ has been calculated, the consumption tracking device 222 may use an algorithm based on various thresholds to determine how to classify subsequent changes in the liquid volume over time. For example, if a volume change is a decrease by an amount greater than the typical human drink volume V(human) within a short period of time T(human), the volume change is likely resulting from the user dumping liquid and is not considered as being consumed by the user. If the volume change is an increase by any amount, the volume change is likely resulting from the user adding liquid to the container. This added height and/or volume amount may be added to the stored height and/or volume, e.g., from the initial filling event less any intervening drinks. If a volume change is a decrease by an amount typical of human drink volume, e.g., falling within predetermined threshold values, then the processor 124 may consider the volume change as being consumed by the user and process and/or store the height and/or volume data.

Alternatively, if the container 208 is of the type described in FIG. 2A, the processor 124 may acquire pressure data from the pressure sensor 219 to measure pressure changes during a user drink event to calculate the volume of air flow through the primary airflow passageway 215, e.g., using Bernoulli's equation as previously described. In this method, the volume of liquid consumed is equal to the volume of air that flowed into the container 208 during the drink event.

Alternatively, the processor 124 may acquire pressure data from the pressure sensor 219 to determine the volume of liquid consumed by subtracting the pressure just before a unique drink start event from the pressure just after a corresponding drink end event. The calculated pressure change may then be used to calculate the liquid volume consumed during the drink event.

To perform this method, the processor 124 determines the drink start and drink end events. If the container 208 is of the type described in FIG. 2A, then the processor 124 may acquire pressure data periodically from the pressure sensor 219 to identify a unique pressure change signature pattern that corresponds to a drink start event and a different unique pressure change signature pattern that corresponds to a drink end event. Alternatively, if the container 208 includes an accelerometer, the processor 124 may acquire motion data from the accelerometer to identify a unique container motion signature pattern that corresponds to a drink start event and a different unique container motion signature pattern that corresponds to a drink end event.

Optionally, any of the systems 200-200″ may also include an accelerometer or other motion sensor (not shown) mounted on the container body 210 or container top 211. The motion sensor may communicate with the processor 124 so that the processor 124 and/or other electronic components may be powered off or placed in a low-power state for the purpose of reducing overall energy consumption of the system 200-200.″ When the processor 124 detects movement of the container 208-208″ based on motion data from the motion sensor, the processor 124 may activate the components of the detection device 222, enabling the device 222 and/or system 200 to transition to a full-power state, e.g., while the container 208-208″ is in motion and/or for a predetermined time, similar to other embodiments herein.

Additionally, the accelerometer may provide an inclinometer that may be used to automatically correct pressure sensor measurements based on simultaneously measured angle of the container 208-208″ relative to vertical. For example, the processor 124 may acquire pressure data from the pressure sensor 219 and orientation data from the accelerometer to determine the orientation of the container 208-208″ corresponding to the pressure data. Thus, the processor 124 may compensate for pressure data acquired while the container 208-208″ is at an angle other than vertical (for example, orientation data indicating the container 208 to be at a thirty degree angle from vertical may be used to increase the actual height of the liquid relative to the pressure data acquired from the pressure sensor 219). By automatically correcting all pressure sensor measurements using the accelerometer, the volume of liquid in the container 208-208″ may be accurately computed largely independent of the position or angle that the user is holding the container 208-208.″ Optionally, the processor 124 may identify that the container 208-208″ is tilted at an angle greater than ninety degrees (90°) or other threshold and discard pressure data acquired at this orientation and/or defer acquiring pressure data until the orientation data indicates the container 208-208″ is between zero and ninety degrees (0-90°) from vertical. Another challenge overcome by the systems 200-200″ is how to measure pressure while the container 208-208″ is in motion. The systems 200-200″ may be used in many real world situations including while sitting, walking, hiking, running, riding a bike, riding in a car, or other common activities. Since most of these activities cause the container 208-208″ to be in motion, the liquid in the container 208-208″ is also in motion and sloshing in the container 208-208,″ which in turn causes unstable pressure measurements while the system is attempting to measure the liquid column pressure. The processor 124 may analyze pressure data from the pressure sensor 219 to distinguish an actual drink event from such other movements.

In addition to the angle adjustment techniques already mentioned, the consumption tracking device 222 may also employ noise identification and filtering algorithms to ignore pressure readings that are not valid and/or to remove random noise and motion induced noise from the pressure measurements. When combined, these techniques may dramatically improve the accuracy and usability of the system, enabling the system 200-200″ to be used in calm or harsh environments.

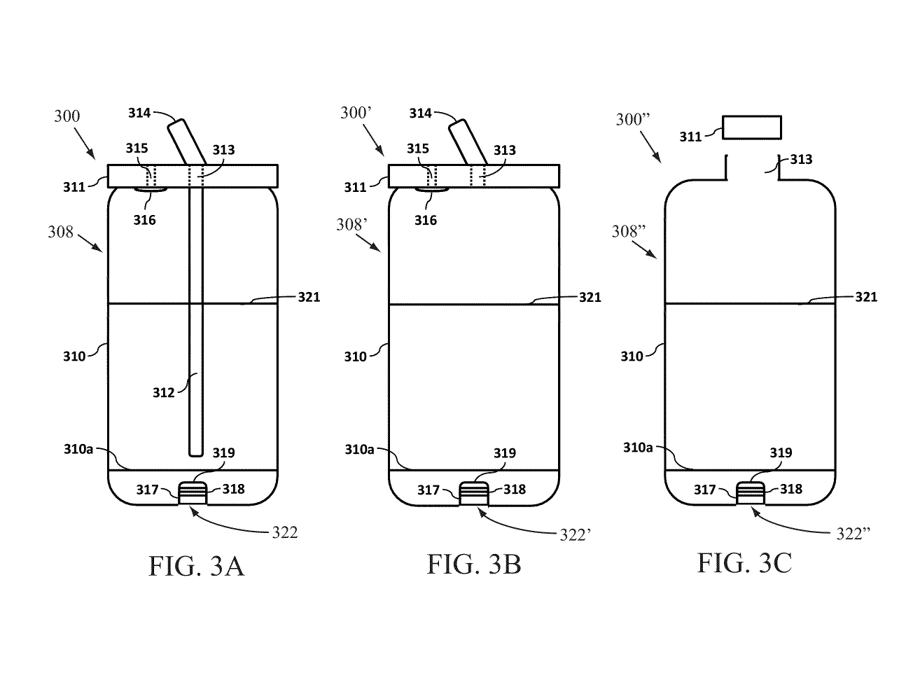

Turning to FIGS. 3A-3C, alternative examples of another hydration tracking system 300-300″ are shown that are generally similar to the systems 100, 200 although using an accelerometer instead of pressure sensors 119 or 219.

For example, FIG. 3A illustrates a liquid dispensing container 308 including a container body 310 having a container floor 310a, and one or more of a removable top 311, straw 312, connecting tube 313, drinking tube (bite or open) 314, primary air flow passageway 315, and/or a one-way air valve 316. The container 308 may be filled with a liquid or other fluid 321 for consumption, which in turn may result in an air chamber 320 above the liquid when the top 311 is placed on the container body 310. A user of the container 308 consumes liquid from the container by drawing liquid through the drinking tube 314.

FIG. 3B illustrates an alternate liquid dispensing container 308′ without a straw member. A user of the container 308′ consumes liquid from the container 308′ by tilting the container 308′ such that liquid is in contact with the drinking tube 314 and then drawing liquid through the drinking tube 314 and/or squeezing the container 308′ to force liquid through the drinking tube 314.

FIG. 3C illustrates another alternate liquid dispensing container 308″ without a straw member or drinking tube. A user of the container 308″ consumes liquid from the container 308″ by tilting the container 308″ such that liquid flows out of the drinking orifice 313.

Each of these hydration tracking systems 300-300″ may also include a liquid consumption tracking device 322 including a mass measurement device, e.g., based on an accelerometer 319 attached to the bottom 310a of the container body 310 below the container floor as shown in FIGS. 3A-3C or otherwise attached to any other part of the container 308-308.″ For example, the accelerometer 319 may be attached to the outside of the container 308-308″ such that it is never in contact with the liquid or air in the container 308-308,″ or may be attached to the inside of the container 308-308″ such that it may come in contact with the liquid or air. The consumption tracking device 322 may also include a replaceable or rechargeable battery 317 and electronic components 318, e.g., similar to the consumption tracking device shown in FIG. 1B including a processor 124, memory 128 for storing data and instructions executable by the processor 124, and/or a wireless communication interface 126 for communicating with an external computer or mobile device (not shown).

Optionally, the consumption tracking device 322 may include one or more additional sensors such as an accelerometer, inclinometer, gyroscope, temperature sensor, and/or humidity sensor. In addition or alternatively, the consumption tracking device 322 may also be electronically connected to one or more LEDs or a visual display (not shown) mounted anywhere on or in the container body 310 or container top 311 to provide various types of feedback to the user of the system 300-300.″

The electronic components of the consumption tracking device 322 may be enclosed in a housing that is designed to easily connect to the container 308-308.″ The housing may be designed to be water proof and dishwasher safe to protect the electronic components from exposure to the liquid in the container 308-308″ and/or to water or steam during cleaning of the hydration tracking system 300-300.″

The processor 124 may acquire motion data from the accelerometer 319 to measure tiny changes in the motion of the container 308-308″ in three dimensional space and these measured changes may be used by a unique mass analysis algorithm to accurately calculate the mass of the container 308-308.″ The mass of the liquid at any point in time may then be determined by subtracting the pre-determined fixed mass of the empty container 308-308″ from the total mass of the container 308-308″ including liquid at the time of data acquisition. The processor 124 may then determine the volume of liquid consumed in any time interval by periodically (or in response to an identified drink event, similar to other embodiments herein) measuring the mass of liquid to calculate the change in liquid volume during the time interval.

Whenever the container 308-308″ is held by a human hand, the container 308-308″ is partially free to move independent of the human hand. This is due to the fact that a human typically does not grasp the container 308-308″ so tightly as to fully restrict the container motion relative to the human motion. Further, since the liquid in the container 308-308″ is not part of the container 308-308″ and can independently move within the container 308-308,″ causing detectable variations in accelerations based only on the liquid motion. These unique observations make it possible to use an accelerometer to measure the motion of the container 308-308″ including the effect of the liquid sloshing in the container 308-308″ in order to determine the total mass of the container 308-308.″

The accelerometer mass estimation approach is based on conservation of inertial periodicity in the presence of a gravitational field, which is similar to a pendulum equation after finding a parametric association of moment distance to liquid/bottle mass assuming a known mass density.

The mass analysis algorithm first identifies specific acceleration characteristics that are independent of the container motion Ai. These acceleration characteristics are determined by examining acceleration variances as the container 308-308″ is in motion, e.g., in real time during use by the processor 124. The mass analysis algorithm then uses multiple difference functions on Ai to identify additional characteristics Am that vary directly as a function of the mass of the container 308-308,″ for example, the sloshing motion of the fluid within the container 308-308.″ Once the Am characteristics have been identified, they can be used to analyze typical container motion in order to accurately predict the mass of the container 308-308.″

Algorithm Description:
a. Subtract macro acceleration components using a method similar to high-filtering.
b. Perform a secondary low-pass filtering process of the residual signal.
c. Identify the relative deviation of the normalized post-filtered mean-deviations.
d. Identify macro kinetic energy from sum of all channel second order variances (variance of the variance).
e. Perform mapping of mass to characteristic periodicity of residual signal.

Additionally, the processor 124 may acquire data from the accelerometer 319 (or other motion sensor on the container 308-308″) to identify drink start and drink end events such that the mass calculation is performed only while the user is drinking, similar to other embodiments herein. This approach may improve the accuracy of the mass calculation.

Similar to other embodiments, the processor 124 may acquire motion data from the motion sensor so that the processor 124 and/or other electronic components may be powered off or placed in a low-power state for the purpose of reducing overall energy consumption of the system 300-300.″ When the processor 124 detects movement of the container 308-308″ based on motion data from the motion sensor, the processor 124 may activate the components of the detection device 322, enabling the device 322 and/or system 300-300″ to transition to a full-power state, e.g., while the container 308-308″ is in motion and/or for a predetermined time, similar to other embodiments herein.

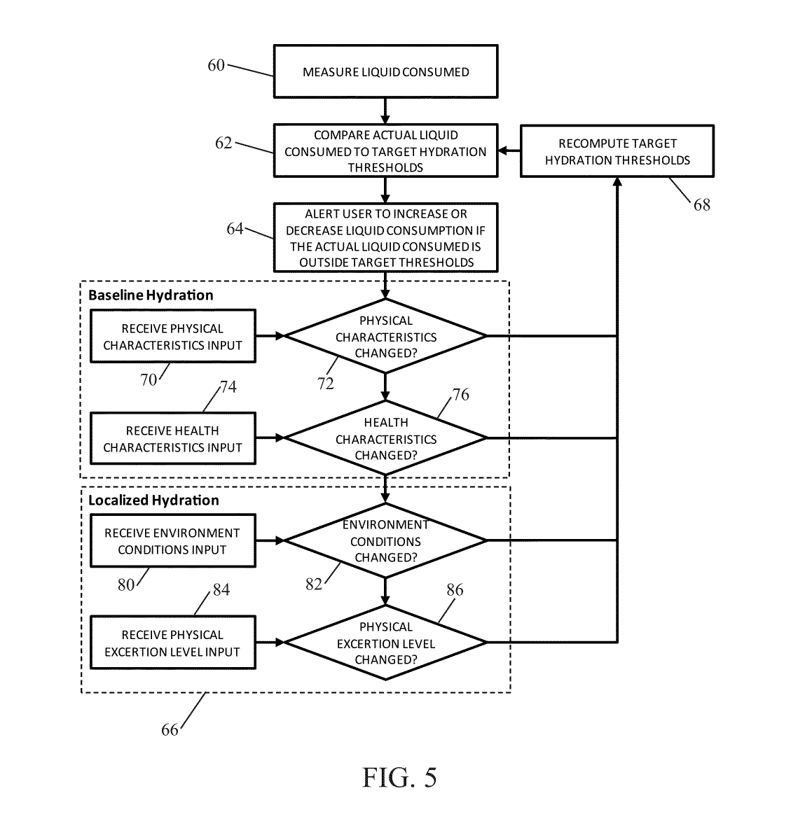

Turning to FIG. 4, any of the fluid containers herein may be incorporated into a hydration and/or fitness management system 10 that includes a fluid container 100 (which may be any of the embodiments herein), an electronic device 18, and a fitness tracker 20, which may be used together by a user 90 to monitor the user's hydration while engaged in various physical activities, such as walking, hiking, running, bicycling, and the like. The electronic device 18 may communicate with the fluid container 100 and fitness tracker 20 wirelessly, e.g., using Bluetooth, radiofrequency and/or other known communication protocols, e.g., to acquire data, perform various functions, and provide recommendations and/or other output to the user 90 during various activities. FIG. 5 illustrates an example of a method that may be used by the system 10 to monitor hydration of the user 90.

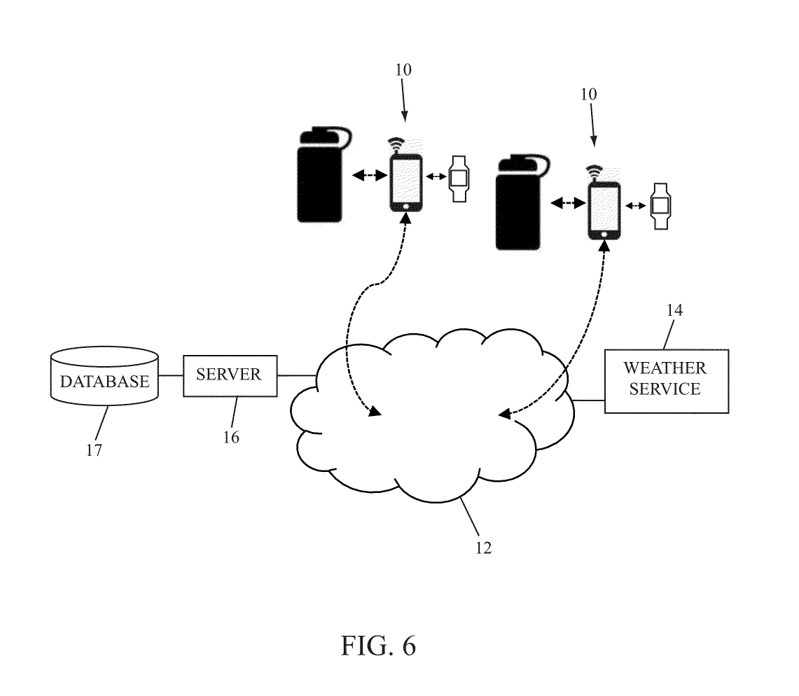

One or more of the steps of the method may be performed by the electronic device 18, e.g., by accessing a database and/or functionality stored in memory of the mobile device 18. Alternatively, as shown in FIG. 6, the electronic device 18 communicate via a network 12 to receive additional data, e.g., environmental conditions from a weather service 14, and/or exchange data with a central server 16 and/or database 17. Optionally, multiple systems 10 (each including an electronic device 18, fluid container 100, and optionally a fitness tracker 20) may communicate with the server 16 and/or database 17 via the network 12, e.g., to share information, allow collection and/or analysis of data related to hydration of multiple users, and the like, as described elsewhere herein.

In exemplary embodiments, the network 12 may be a telecommunications network, including a wide area network (“WAN”), a local area network (“LAN”), an intranet, a wireless network, and/or a telephony network. For example, the network 10 may incorporate several different types of networks including a WAN, a LAN, and/or a wireless network; one such network including multiple different types of networks is the Internet.

The central server 16 may include one or more computer systems including one or more processors, memory and/or storage devices, and communication interfaces for communicating via the network 12, e.g., with the electronic devices 18. The central server 16 may include one or more hardware-based components and/or software-based modules for performing the various functions related to the system 10, as described elsewhere herein. Although only one vendor server 16 and database 17 are shown, it will be appreciated that multiple servers and/or databases (not shown) may be provided at the same or different locations for performing the various functions described herein.

The fitness tracker 20 may include any device that monitors one or more characteristics of the user's activities. For example, the fitness tracker 20 may be a GPS device that monitors movement of the user, an electronic pedometer or other device that detects steps and/or other motion to estimate activities, a heart rate monitor, and the like, such as those available from Garmin, Fitbit, and the like.

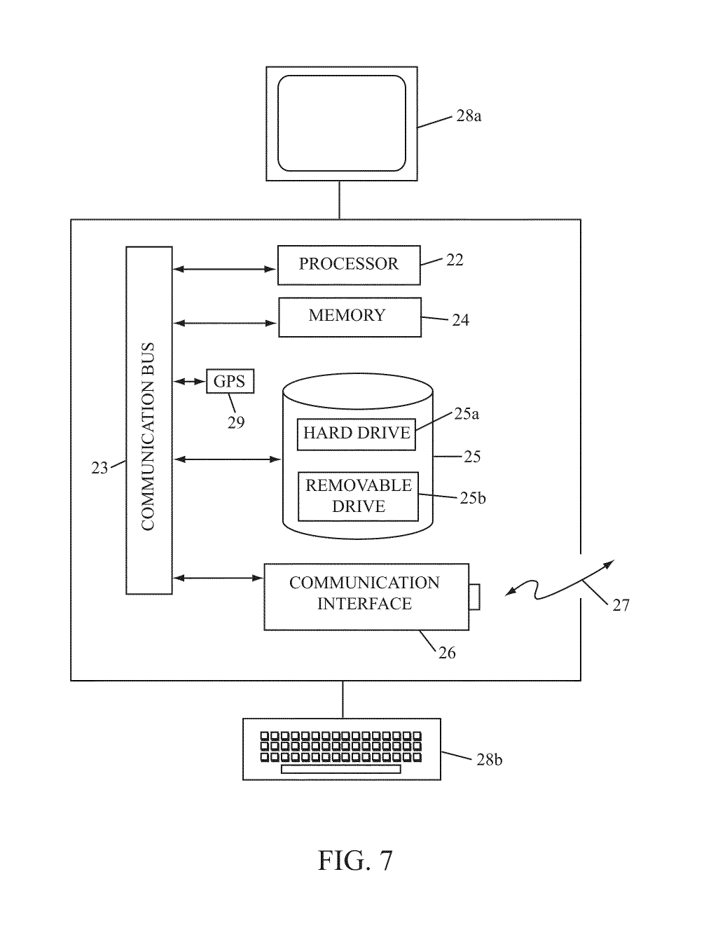

Turning to FIG. 7, an exemplary embodiment of an electronic device 18 is shown that includes one or more hardware and/or software components for performing the methods described herein. As shown, the electronic device 18 may be a wireless device, e.g., a mobile, smart, and/or cellular telephone, a tablet computer, a personal digital assistant, a Wi-Fi device, a laptop computer, and the like, capable of communicating via the network 12 (not shown, see FIG. 6). The electronic device 18 includes one or more processors, such as exemplary processor 22, for completing the various tasks described herein, e.g., to acquire data from the sensors of the fluid container 100 to determine fluid consumption of the user, to acquire data from the fitness tracker 20 to determine exertion levels of the user, and/or to provide recommendations to the user, as described elsewhere herein. Additional processors may be provided, such as an auxiliary processor to manage input/output or perform floating point mathematical operations, a special-purpose microprocessor having an architecture rapid execution of signal processing algorithms, a slave processor subordinate to the main processing system (“back-end processor”), and/or a coprocessor (not shown). Such auxiliary processors may be discrete processors or may be integrated with the processor 22 and collectively may still be referred to as “a processor.”

The processor 22 is generally connected to a communication bus 23. The communication bus 23 may include a data channel for facilitating information transfer between storage and/or other components of the electronic device 18. The communication bus 23 may also provide signals required for communication with the processor 22, including a data bus, address bus, and/or control bus (not shown). The communication bus 23 may include any known bus architecture, for example, industry standard architecture (ISA), extended industry standard architecture (EISA), Micro Channel Architecture (MCA), peripheral component interconnect (PCI) local bus, IEEE 488 general-purpose interface bus (GPIB), IEEE 696/S-100, and the like.

The electronic device 18 also includes memory and/or storage devices, e.g., main memory 24 and secondary memory or storage devices 25. The main memory 24 may provide storage of instructions and/or data for programs executed on the processor 22. In exemplary embodiments, the main memory 24 may be semiconductor-based memory, such as dynamic random access memory (DRAM) and/or static random access memory (SRAM). In addition, other semiconductor-based memory may also be provided, such as synchronous dynamic random access memory (SDRAM), Rambus dynamic random access memory (RDRAM), ferroelectric random access memory (FRAM), and the like, as well as read only memory (ROM).

The secondary memory 25 may include a hard disk drive 25a and/or a removable storage drive 25b, for example, a flash drive, a floppy disk drive, a magnetic tape drive, an optical disk drive, a CDROM drive, a DVDROM drive, and the like (not shown). The removable storage drive 25 may read from and/or write to a removable storage unit (not shown) in a well-known manner. In exemplary embodiments, the removable storage unit may include a floppy disk, magnetic tape, optical disk, CDROM disk, DVDROM disk, and the like that may be read from and/or written to by removable storage drive 25b. Additionally, the removable storage unit may include a computer usable storage medium with computer software and computer data stored thereon.

Optionally, the secondary memory 25 may include other components allowing computer programs and/or other instructions to be loaded into the electronic device 18. For example, such components may include semiconductor-based memory such as programmable read-only memory (PROM), erasable programmable read-only memory (EPROM), electrically erasable read-only memory (EEPROM), or flash memory (block oriented memory similar to EEPROM). Also included are any other interfaces and removable storage units that allow software and data to be transferred from the removable storage unit to the electronic device 18.

The electronic device 18 also generally includes one or more communication interfaces 26, e.g., one or more transceivers, receivers, and/or transmitters. Communication interface(s) 26 may allow software and/or data to be transferred between the electronic device 18 and the fluid container 100, fitness tracker 20, a weather service 14, central server 16 and/or other external devices, networks, or information sources. Examples of communication interface 26 include but are not limited to an infrared or radiofrequency (“RF”) interface (such as those that use the Bluetooth standard), a modem, a network interface (for example an Ethernet card), a communications port, a PCMCIA slot and card, and the like. The communication interface(s) 26 may implement industry promulgated architecture standards, such as Ethernet IEEE 802 standards, Fibre Channel, digital subscriber line (DSL), asymmetric digital subscriber line (ASDL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and the like. Software and/or data transferred via the communication interface 26 may be transferred using signals 27, such as electronic, electromagnetic, optical signals, and the like. The signals 27 may be implemented using wires, cables, fiber optics, telephone lines, cellular phone links, radio frequency (RF) links, and/or other communications channels.

Computer programming instructions, e.g., computer programs, software, or firmware, may be stored in the main memory 24 and/or the secondary memory 25. Computer programs may also be received via the communication interface 26. Such computer programs, when executed, may enable the electronic device 18 to perform one or more of the features described elsewhere herein.

As used herein, “computer program product” may refer to any media used to provide programming instructions to the electronic device 18. Examples of such media include removable storage units in removable storage drive 25b, a hard disk installed in hard disk drive 25a, and signals 27. Thus, a computer program product may include means for providing programming instructions to the electronic device 18.

Where the methods and/or features described herein are completed using software, the software may be stored in a computer program product and loaded into the electronic device 18, e.g., using the hard disk drive 25a, removable storage drive 25b, and/or communication interface 26. The computer programming instructions, when executed by the processor 22, may cause the processor 22 to perform the methods and/or features described herein. In addition or alternatively, one or more of the methods and/or features may be implemented primarily in hardware using hardware components, such as application specific integrated circuits (“ASICs”).

In addition, the electronic device 18 may include one or more user interfaces 28, e.g., a keyboard 28b, mouse, touch screen, touch pad (not shown), and/or other input device. The user interface 28 may allow a person using the electronic device 18 to download software, launch one or more programs, and the like, as described elsewhere herein.

Further, the electronic device 18 may include one or more output devices, e.g., a display 28a, and the like. The output device(s) 28a may facilitate a user controlling and/or otherwise communicating with the processor 22 or other components of the electronic device 18. In addition, the output device(s) 28a may allow information to be presented and/or manipulated in a desired manner, as described elsewhere herein. In one embodiment, the electronic device 18 may include a touch screen (not shown) that may act as a display 28a and as an input device 28b, allowing the user to scroll through menus and/or select icons, e.g., by touching the corresponding images on the touch screen, as described elsewhere herein.

Optionally, the electronic device 18 may include one or more additional hardware components and/or software modules. For example, the electronic device 18 may include a GPS 29 or other device or system for identifying a location of the electronic device 18, e.g., to facilitate identifying the user's location.

Returning to FIGS. 4-6, the system 10 may be used to track the volume of liquid consumed from a container 100 over time in order to determine the hydration level of the user 90 and/or to provide recommendations to the user. The system 10 may be further enhanced with real-time environmental condition data and real-time physical exertion data about a user to create a closed loop hydration tracking system. For example, the fitness tracker 20 and/or the electronic device 18 may include a GPS device, which may provide the user's location, and the electronic device 18 may communicate with a weather service 14 to provide environmental conditions at the user's location that may affect the desired hydration level of the user, e.g., temperature, humidity, how sunny/cloudy the location is, and the like. Alternatively, environmental conditions may be acquired directly from one or more sensors, e.g., a temperature and/or humidity sensor on the fluid container 100 or other component of the system, as described elsewhere herein.

For example, with particular reference to FIG. 5, the hydration level of the user at any time may be compared to a hydration plan specific to the user to determine if the user is within desired hydration thresholds. As shown, the thresholds may be set and/or modified based on a “baseline hydration plan,” which may be derived from predetermined physical and/or health characteristics of the user and/or a “localized hydration plan, which may be based on environmental conditions and/or physical activity of the user.

Generally, the processor 22 of the electronic device 18 (or optionally the consumption tracking device carried by the fluid container 100 itself) may perform the substantially continuous loop shown in FIG. 5 once the desired devices are activated. For example, initially, the user may activate a hydration application on the electronic device 18, the fitness tracker 20, and/or the consumption tracking device on the fluid container 100, e.g., immediately before engaging in a desired activity, such as walking, hiking, running, bicycling, and the like. Alternatively, the devices may be activated to monitor the user's hydration at any time, e.g., from the beginning and during the course of an ordinary day.

When the container 100 is filled (and/or subsequently refilled), the consumption tracking device may identify and store fluid data (e.g., the volume of fluid provided in the container 100) and/or communicate the fluid data to the electronic device 18. Thereafter, at step 60 in FIG. 5, the consumption tracking device may measure fluid consumed by the user, e.g., as described elsewhere herein with reference to any of the fluid containers, which may be stored by the consumption tracking device and/or communicated to the electronic device 18.

At step 62, the actual consumed liquid may be compared to target thresholds, e.g., established by the application running on the electronic device 18. If the user is determined to be outside of desired hydration thresholds, at step 64, the electronic device 18 may notify or alert the user in real-time to drink more or less fluid, e.g., via one or more output devices on the fluid container 100, or on the display 28a of the electronic device 18. At steps 66-68, the application may set and/or recalculate the target hydration thresholds, e.g., based on one or more characteristics of the user and/or their environment.

For example, the baseline hydration characteristics 70 (shown in FIGS. 4 and 5) may be initially set by the user when the application is installed on the electronic device 18. For example, the application may prompt the user to enter standard characteristics, such as gender, age, race, height, and/or weight. Similarly, the application may prompt the user to provide relevant health characteristics 74, e.g., general healthiness, current health conditions, amount of sleep, heart rate, and the like. Alternatively, such characteristics may be provided automatically, e.g., based on the information from other applications or the fitness tracker 20.

Generally, these characteristics remain substantially static, although during the recalculation step 66, e.g., at regularly scheduled intervals, the application may prompt the user to enter any changes at steps 72, 76, or the application may automatically update such changes.

Optionally, the baseline hydration plan may be further enhanced with real-time local environmental conditions, at steps 80, 82, including temperature, humidity, or altitude, and/or further enhanced when combined with real-time measured physical activity of the user at steps 84, 86. This enhanced “localized hydration plan” may be uniquely personalized and updated in a closed-loop real-time feedback system to more accurately measure the actual hydration requirements of a user and set new hydration thresholds. At step 68, the new hydration thresholds may replace the previous thresholds and used in subsequent comparisons with the user's actual fluid consumption.

In one embodiment, at step 80, if the container 100 includes temperature and/or humidity sensors, the application on the electronic device 18 may acquire temperature and/or humidity data to measure the real-time environmental conditions and check for changes at step 82 in order to enhance the baseline hydration plan, e.g., increasing the recommended volume of fluid as temperature increase. Alternatively, the environmental conditions may be acquired from a weather service 14, as described previously.

Further, at step 66, the application on the electronic device may communicate with the fitness tracker 20, e.g., to measure physical energy expended during a time interval and/or other exertion parameters. At step 84, the physical energy expended during a time interval may be measured in various units including calories burned, heart rate patterns, and/or other units of measure based at least in part on the data provided by the fitness tracker 20. Any changes in physical activity may be updated at step 86 and used to automatically and dynamically adjust hydration targets and thresholds.

Thus, the hydration tracking system 10 shown in FIG. 4 may provide a unique coupling of a hydration tracking device, environmental measurements, and physical exertion information to implement a feedback loop wherein processor algorithms may update both the hydration plan and target hydration thresholds to more accurately predict the user's hydration requirements in real-time. By more accurately tracking the user's hydration requirements and current status, the system 10 may more accurately notify the user when to drink or when not to drink in order to maintain proper hydration.

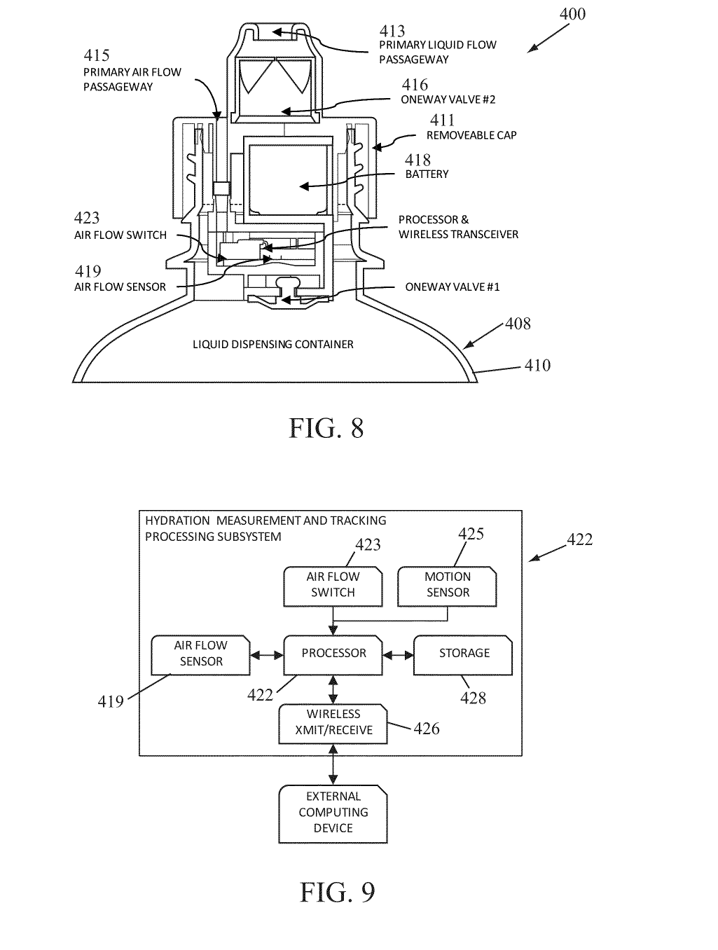

Turning to FIGS. 8-10, another exemplary embodiment of a device and system 400 for monitoring fluid consumption are shown that generally include a fluid container 408 and a consumption tracking device 422, similar to other embodiments herein. However, unlike previous embodiments the consumption tracking device 422 includes an air flow sensor 419 and an air flow switch 423.

In particular, as shown, the fluid container 408 includes a container body 410 and a removable top 411 including a liquid flow passageway 413, an air flow passageway 415, and a one-way air valve 416. The container 408 may be filled with a liquid or other fluid for consumption, which in turn may result in an air chamber above the liquid when the top 411 is placed on the container body 410. A user of the container 408 consumes liquid from the container 408 by drawing liquid through the liquid flow passageway 413, thereby causing air to flow into the container 408 through the air flow passageway 415, opening the air valve 416, similar to other embodiments. However, unlike the previous embodiments, the air flow sensor 419 and air flow switch 423 communicate with the air flow passageway 415, e.g., to detect and measure air flow through the air flow passageway 415.

In addition, the system 400 includes a liquid consumption tracking device 422 coupled to the air flow sensor 419 and air flow switch 423 that includes a processor 424, memory 428 for storing data and instructions executable by the processor 424, and a wireless communication interface 426 for communicating with a remote device (not shown).

By sealing the liquid dispensing container 408 and providing a controlled air flow passageway 415 for air to enter the container 408 as liquid is flowing out of the container 408 through the liquid flow passageway 413, the processor 424 may acquire flow data from the air flow sensor 419 to measure the amount of air that is flowing into the container 408 during a liquid consumption event. The volume of air flowing into the container 408 is displacing the liquid flowing out of the container 408 and is proportional to the volume of liquid flowing out of the container 408. In alternative embodiments, the air flow sensor 419 may be replaced with one or more different sensors, such as any of those described elsewhere herein.

The air flow switch 423 may be a very low power device coupled to the processor 424 such that the processor 424 may detect whenever the slightest air flow is detected in the air flow passageway 415. Because of the presence of the air flow switch 423, the other components of the system 400 may be transitioned into a low-power state while there is no air flow detected, thereby saving significant power consumption in the overall system over time. Thus, the air flow switch 423 may dramatically reduce power consumption of the system 400 and therefore dramatically improve usability.

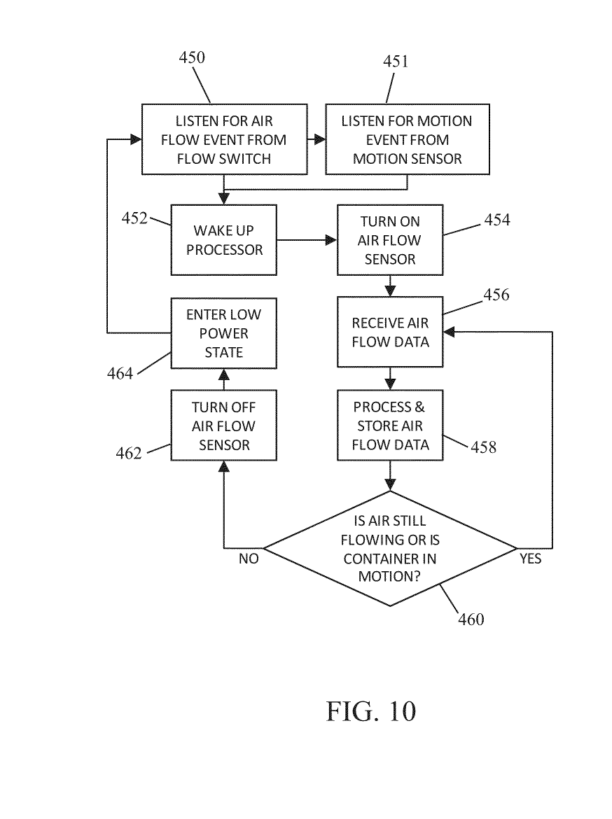

FIG. 10 shows an exemplary method for using the system 400 of FIGS. 8 and 9. In an exemplary embodiment, initially, the system 400 may be in a low-power state. At step 450, the processor 424 may acquire data from the air flow switch 423 to identify an air flow event. In addition or alternatively, at step 451, the processor 424 may acquire motion data from a motion sensor (not shown) to identify a potential consumption event, similar to other embodiments herein.

Once such an event is identified, at step 452, the system may be awakened and the air flow sensor 419 may be turned on. The processor 424 may then acquire air flow data at step 456 and process and/or store the data at step 458. At step 460, this process may be repeated until air is now flowing through the air flow passage 415, whereupon the processor 424 may turn off the air flow sensor 419 at step 462 and the system 400 may reenter the low-power state at step 464 in preparation for another event.

Turning to FIG. 6, once a given embodiment of a system measures the volume of liquid consumed from an individual liquid dispensing container, the next major challenge is to communicate hydration and other locally collected data to remote device, such as electronic device 18 located near the liquid dispensing container 100; and to a central server 16. In order to improve usability, this communication channel may be implemented using wireless technology such as Bluetooth or WiFi. If the server 16 is collecting data from a plurality of liquid dispensing containers 100, the server 16 may perform analysis that is not possible with data only from a single liquid dispensing container 100. Such analysis may include providing hydration information across a population of users or identifying trends by combining hydration data stored in the database 17 at the server 16 with any external data source including time, season, weather, geography, population, demographics, income, social networks, or any other data that is external to the data collected from the liquid dispensing containers.

Optionally, in any of the embodiments herein, the container, e.g., the top or container body, may include a connector, e.g., a USB connector, for coupling the battery and/or other power source for the consumption tracking device to an external energy source, e.g., electrical outlet, computer, and the like.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for monitoring hydration, comprising:
a fluid container comprising a container body defining an interior region and a removable top for enclosing the interior region;
a one-way valve in the top biased to a closed position for sealing an opening in the top communicating with an exterior of the fluid container and movable to an open position when the interior region experiences a negative pressure to allow air to flow into the interior region from the exterior via the opening;
a drinking tube extending from the top into interior region;
an air pressure sensor within the fluid container at a location such that the sensor is exposed to air when the fluid container is filled with a hydration fluid; and
a processor coupled to the sensor and configured to detect a first predetermined pressure change within the interior region corresponding to a drink start event when a user begins to drink hydration fluid from the interior region via the drinking tube,
the processor further configured to determine a volume of hydration fluid consumed by the user based on changes in air pressure within the interior region.

2. The device of claim 1, wherein the processor is further configured to detect a second predetermined pressure change within the interior region corresponding to a drink end event when the user discontinues drinking hydration fluid from the interior region via the drinking tube.

3. The device of claim 1, further comprising memory communicating with the processor for storing consumption data determined by the processor.

4. The device of claim 1, further comprising a communication interface communicating with the processor for communicating consumption data determined by the processor to a remote device.

5. The device of claim 1, further comprising a temperature sensor on the fluid container, the processor coupled to the temperature sensor for determining air temperature of air entering the interior region when the one-way valve is opened, the process configured to calculate the volume of hydration fluid consumed based at least in part on the determined air temperature.

6. The device of claim 1, further comprising a humidity sensor on the fluid container, the processor coupled to the humidity sensor for determining humidity of air entering the interior region when the one-way valve is opened, the process configured to calculate the volume of hydration fluid consumed based at least in part on the determined humidity.

7. The device of claim 1, further comprising a motion sensor coupled to the processor, the processor configured to operate in a first, low-power state until the motion sensor provides a signal indicating that the fluid container is moving, whereupon the processor is configured to activate in a second state anticipating a drink start event.

8. The device of claim 1, further comprising an output device on the fluid container, the processor coupled to the output device for presenting information related to the user's hydration.

9. The device of claim 1, wherein the air pressure sensor is mounted to the top at a location spaced apart from the one-way valve.

10. (canceled)

11. A device for monitoring hydration, comprising:
a fluid container comprising a container body including a side wall and a bottom wall defining an interior region and a removable top for enclosing the interior region;
a drinking port in the top for consuming hydration fluid contained within the fluid container;
a liquid pressure sensor mounted to the bottom wall within the interior region;
an inclinometer mounted to the fluid container; and
a processor coupled to the pressure sensor to acquire pressure data to determine a height of hydration fluid within the interior region and coupled to the inclinometer to acquire orientation data to determine an angular orientation of the fluid container when the height is determined, the processor further configured to determine a volume of hydration fluid consumed by the user based on changes in the height of the hydration fluid within the interior region corrected by the angular orientation.

12. The device of claim 11, wherein the processor is configured to identify a fill event when the fluid container is filled with hydration fluid based on a predetermined change in pressure data from the pressure sensor, the processor configured to determine an initial volume of the hydration fluid based on pressure data and orientation data immediately after identifying the fill event.

13. The device of claim 11, wherein the processor is configured to identify a first drink event when a user first consumes hydration fluid from the fluid container, the processor configured to a remaining volume of the hydration fluid based on pressure data and orientation data immediately after identifying the first drink event, the processor configured to determine the volume of hydration fluid consumed based on the difference between the initial volume and the remaining volume.

14. The device of claim 11, further comprising a motion sensor coupled to the processor, the processor configured to operate in a first, low-power state until the motion sensor provides a signal indicating that the fluid container is moving, whereupon the processor is configured to activate in a second state anticipating a drink event.

15. The device of claim 11, wherein the inclinometer comprises an accelerometer, and wherein the processor is configured to operate in a first, low-power state until the accelerometer provides a signal indicating that the fluid container is moving, whereupon the processor is configured to activate in a second state anticipating a drink event.

16. The device of claim 11, further comprising a motion sensor coupled to the processor, the processor configured to acquire motion data from the motion sensor to identify a drink event and determine a change in volume of the hydration fluid based on changes in the height of the hydration fluid air pressure within the interior region corrected by the angular orientation.

17-27. (canceled)

28. A method for monitoring hydration of a user of a fluid container including a motion sensor, comprising:
receiving a signal from the motion sensor indicating that the container is in motion or a drink event has begun;
based on the signal, transitioning electronic components of the consumption tracking system from a low-power state to a full-power state; and
acquiring sensor data from one or more sensors on the fluid container and determining, at the consumption tracking system, an amount of fluid consumed from the fluid container during the drink event based at least in part on the sensor data.

29-50. (canceled)

51. A system for managing hydration of a user, comprising:
a fluid container comprising one or more sensors for identifying when the user consumes fluid from the container and a container communication interface;
a fitness tracker for monitoring one or more aspects of the user's physical activity; and
a mobile device comprising a mobile device communication interface for communicating with the one or more sensors of the fluid container via the container communication interface to monitor at least one of volume and frequency of consumption of hydration fluid from the container and for communicating with the fitness tracker to monitor one or more aspects of the user's physical activity, and a mobile device processor configured to monitor hydration of the user based at least in part on the hydration fluid consumed from the container and the user's physical activity and provide an output on an output device to the user related to compliance with hydration recommendations.

52. The system of claim 51, wherein the one or more sensors comprise a motion sensor and a pressure sensor, the container further comprising a container processor coupled to the motion sensor to identify a drink event when the user consumes fluid from the container and coupled to the pressure sensor for acquiring consumption data to determine a volume of fluid consumed from the container during the drink event, the container processor communicating volume of fluid consumed data to the mobile device via a container communication interface.

53-54. (canceled)

55. The system of claim 51, wherein the one or more sensors comprise a motion sensor and a pressure sensor, the container further comprising a container processor coupled to the motion sensor to identify a drink event when the user consumes fluid from the container and coupled to the pressure sensor for acquiring consumption data related to the volume of fluid consumed from the container during the drink event, the container further comprising a container communication interface for transmitting the consumption data to the mobile device.

56-61. (canceled)

62. A method for monitoring hydration of a user of a fluid container including one or more sensors and fluid within an interior region of the container, comprising:
receiving one or more signals from the one or more sensors indicating that a drink event has occurred;
acquiring height data from the one or more sensors on the fluid container related to a height of fluid within the container;
acquiring orientation data from the one or more sensors on the fluid container to determine an angular orientation of the fluid container when the height data is acquired; and
determining a height of fluid within the interior region based at least in part on the height data corrected by the angular orientation and determining a volume of fluid consumed during the drink event based on changes in the height of the hydration fluid within the interior region.

63-91. (canceled)
